(19) United States
(12) Patent Application Publication
(10) Pub. No.: US 2021/0375461 A1
(43) Pub. Date: Dec. 2, 2021

(54) MEDICAL DIAGNOSIS SUPPORT SYSTEM, MEDICAL DIAGNOSIS SUPPORT PROGRAM, AND MEDICAL DIAGNOSIS SUPPORT METHOD

(71) Applicant: KONICA MINOLTA, INC., Tokyo (JP)

(72) Inventor: Hiroaki MATSUMOTO, Yokohama-shi (JP)

(21) Appl. No.: 17/333,819

(22) Filed: May 28, 2021

(30) Foreign Application Priority Data
May 29, 2020 (JP) 2020-093855

(51) Int. Cl. G16H 50/20; G16H 50/30; G06T 7/00

(52) U.S. Cl. CPC G16H 50/20; G16H 50/30; G06T 7/0012; G06T 2207/30061; G06T 2207/20081; G06T 2207/20084; G06T 2207/10116

(57) ABSTRACT

A medical diagnosis support system including a hardware processor configured with a program to perform operations including: operation as an acquisition part configured to acquire lifestyle habit information of a subject; operation as an estimation part configured to estimate, using a trained identifier, future lung function of the subject from information on the subject, the information on the subject including the lifestyle habit information acquired from the acquisition part; and operation as a control part configured to control an output part to output a result that has been estimated by the estimation part.

### CROSS REFERENCE TO RELATED APPLICATIONS

This present invention claims priority under 35 U.S.C. § 119 to Japanese Patent Application No. 2020-093855, filed May 29, 2020, the entire content of which is incorporated herein by reference.

### BACKGROUND

### Technological Field

The disclosure relates to a medical diagnosis support system, medical diagnosis support program, and medical diagnosis support method.

### Description of the Related Art

Development of technologies concerning medical support has been active in recent years. For example, Patent Literature 1 (Japanese Unexamined Patent Application Publication No. 2018-113042) discloses a device that compares (a) information on a lifestyle habit and various medical test data of a subject with (b) known, various medical test data related to said lifestyle habit in order to predict future medical test data when said lifestyle habit remains the same.

The disclosure of Patent Literature 1 is limited mainly to health guidance for metabolic syndrome and does not disclose medical diagnosis support for a pulmonary disorder such as asthma or COPD (chronic obstructive pulmonary disease). Thus, there has remained a need for medical diagnosis support concerning the lung function of a subject.

### SUMMARY

In view of the above circumstances, the object of the disclosure is to realize support for a medical diagnosis concerning the lung function of a subject.

To achieve the abovementioned object, a medical diagnosis support system according to a first aspect of the disclosure includes a hardware processor configured with a program to perform operations including: operation as an acquisition part configured to acquire lifestyle habit information of a subject; operation as an estimation part configured to estimate, using a trained identifier, future lung function of the subject from information on the subject, the information on the subject including the lifestyle habit information acquired from the acquisition part; and operation as a control part configured to control an output part to output a result that has been estimated by the estimation part.

According to a fifth aspect of the disclosure, a non-transitory computer readable storage medium stores a medical diagnosis support program that is executable by a computer and that causes the computer to perform operations of a medical diagnosis support system, the operations including: acquiring lifestyle habit information of a subject; estimating, using a trained identifier, future lung function of the subject from information on the subject, the information on the subject including the lifestyle habit information that has been acquired; and controlling an output part to output a result that has been estimated.

According to a ninth aspect of the disclosure, a medical diagnosis support method of a medical diagnosis support system includes: acquiring lifestyle habit information of a subject; estimating, using a trained identifier, future lung function of the subject from information on the subject, the information on the subject including the lifestyle habit information that has been acquired; and controlling an output part to output a result that has been estimated.

### BRIEF DESCRIPTION OF THE DRAWINGS

The advantages and features provided by one or more embodiments of the invention will become more fully understood from the detailed description given hereinbelow and the appended drawings. The drawings are given for the purpose of illustration only and are not intended as a definition of the limits of the present invention.

FIG. 1 is a block diagram showing an example of a functional configuration of a medical diagnosis support system in accordance with at least one embodiment.

FIG. 2 is a flow chart showing an example of processing in accordance with at least one embodiment.

FIG. 3A and FIG. 3B are examples of X-ray images of a subject for Specific Example 1. FIG. 3A is a current X-ray image, and FIG. 3B is a future X-ray image.

FIG. 4 is an example of a spirogram for Specific Example 2.

FIG. 5 is an example of a diagnostic reference chart for Specific Example 3.

FIG. 6 is an example of a flow curve for Specific Example 4.

FIG. 7 is a block diagram showing an example of hardware configuration of a computer for achieving a function of the medical diagnosis support system.

### DETAILED DESCRIPTION OF EMBODIMENTS

Hereinafter, one or more embodiments of the disclosure will be described with reference to the drawings. However, the scope of the disclosure is not limited to the disclosed embodiments.

Note that a common reference symbol is used to describe identical components, and repeat description of such components are omitted.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### Configuration

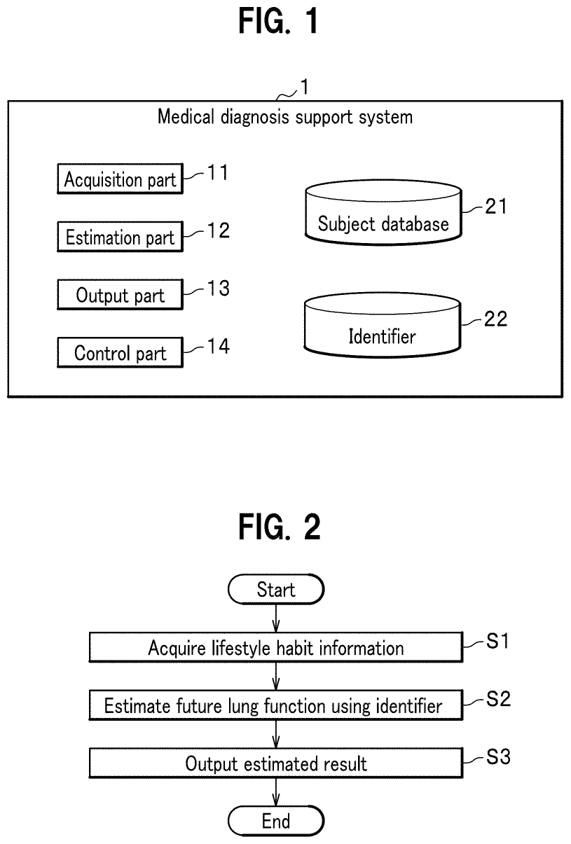

The medical diagnosis support system according to an embodiment is a computer that supports medical diagnosis concerning the lung function of a subject. As shown in FIG. 1, the medical diagnosis support system 1 according to an embodiment includes an acquisition part 11, an estimation part 12, an output part 13, a control part 14, a subject database 21, and an identifier 22.

The acquisition part 11 is configured to acquire one or more types of information regarding a subject. For example, the one or more types of information regarding a subject may be but is not limited to (a) information on a lifestyle habit of the subject; (b) X-ray image information (e.g., an X-ray image of the chest) of the subject obtained using X-ray diagnostic equipment or a diagnostic result (e.g., an amount of change in lung field density, an amount of lung field deformation, or an amount of heart deformation); (c) image information from one or more types of modalities (e.g., CT [computed tomography], MRI [magnetic resonance imaging], or ultrasonic diagnostic equipment) or a diagnostic result; (d) physical information of the subject; or (e) any combination thereof.

The lifestyle habit information of a subject may for example be, but is not limited to, (a) information on a smoking habit of the subject (e.g., the number of cigarettes smoked in a day, the number of years of smoking), (b) a result of spirometer measurement, (c) information on a diet or exercise, or (d) any combination thereof. The physical information of a subject may for example be, but is not limited to, (a) age, (b) gender, (c) height, (d) weight, or (e) any combination thereof. The lifestyle habit information of a subject may be information based on the physical information of the subject. The various types of information regarding the subject may be stored in the subject database 21.

The estimation part 12 is configured to estimate future lung function of a subject from information acquired by the acquisition part 11. The estimation part 12 may, for example, perform the estimation using an identifier 22 that has undergone training.

The output part 13 is configured to output a result (an “estimated result”) that has been estimated by the estimation part 12. An estimated result outputted by the output part 13 may for example be but is not limited to (a) a spirogram, (b) a diagnostic reference chart, (c) a flow curve (a flow-volume curve), or (d) a predetermined type of image. The output part 13 may output at least one of (a) a spirogram, (b) a diagnostic reference chart, (c) a flow curve, or (d) a predetermined type of image. The result outputted by the output part 13 may be stored in the subject database 21.

The control part 14 is configured to control various types of process that the medical diagnosis support system 1 performs. For example, the control part 14 may control the output part 13 to output a result that has been estimated by the estimation part 12.

The subject database 21 is configured to store therein various types of information that are used to support medical diagnosis. In the subject database 21, the information that is acquired by the acquisition part 11 and the estimated result outputted by the output part 13 are stored for each subject. Note that the subject database 21 may be external to the medical diagnosis support system 1, in which case the medical diagnosis support system 1 acquires information from the external subject database 21 as necessary.

The identifier 22 is a trained learning model that has been created with predetermined machine learning using predetermined input data and predetermined output data. Machine learning may for example be, but is not limited to, deep learning, a neural network, or a support-vector machine.

For example, the identifier 22 may be created through machine learning using (a) lifestyle habit information of a COPD sufferer prior to COPD as input data, and (b) a result of spirometer measurement of the same COPD sufferer after the onset of COPD as output data. For example, the identifier 22 may be created through machine learning using more generalized data, with (a) lifestyle habit information of a healthy person of a certain gender, age bracket, and height used as input data, and (b) a result of spirometer measurement of a COPD sufferer of the same gender, age bracket, and height used as output data.

For example, the identifier 22 may be created through machine learning using (a) information on a COPD sufferer including lifestyle habit information prior to COPD and X-ray image information prior to COPD as input data, and (b) X-ray image information of the same COPD sufferer after the onset of COPD as output data. For example, the identifier 22 may be created through machine learning using more generalized data, with (a) lifestyle habit information of a healthy person of a certain gender, age bracket, and height and X-ray image information of a health person of the same gender, age bracket, and height used as input data, and (b) X-ray image information of a COPD sufferer of the same gender, age bracket, and height used as output data.

Instead of using X-ray image information as output data for machine learning, an image processing parameter may be used. Instead of using X-ray image information as output data for machine learning, both an image processing parameter and X-ray image information may be used. The image processing parameter may for example be but is not limited to (a) an amount of change in lung field density, (b) an amount of lung field deformation, (c) an amount of heart deformation, or (d) any combination thereof. The output part 13 may generate a future X-ray image from a current X-ray image using image processing based on an amount of change in lung field density, an amount of lung field deformation, an amount of heart deformation, or any combination thereof that has been used as output data.

### Processing

Processing that is performed by the medical diagnosis support system 1 is described with reference to FIG. 2.

In step S1, the acquisition part 11 acquires lifestyle habit information of a subject. In step S2, the estimation part 12 estimates future lung function of the subject using a trained identifier 22 with the lifestyle habit information acquired by the acquisition part 11 as input. In step S3, the output part 13 outputs a result estimated by the estimation part 12. The output of the output part 13 may be in a desired form.

According to the processing of FIG. 2, the medical diagnosis support system 1 can indicate to a subject an estimated result of future lung function in a case where a lifestyle habit that is indicated by the lifestyle habit information of the subject is maintained.

### Specific Example 1

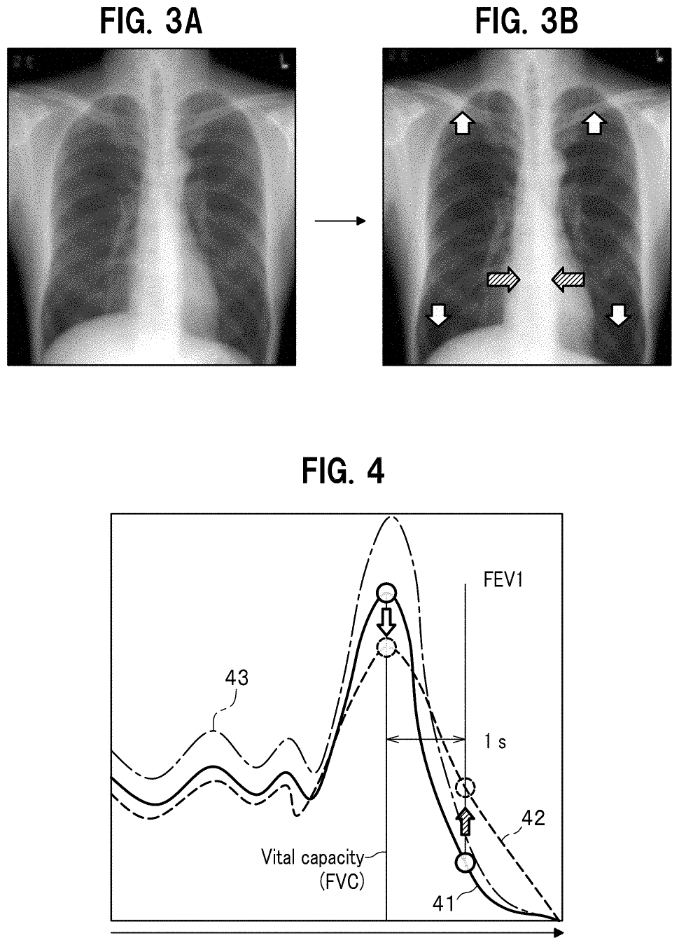

In one example, the acquisition part 11 acquires lifestyle habit information of a subject who is currently healthy and an X-ray image of the subject. The lifestyle habit information is either information on past and present smoking history of the subject or a result of spirometer measurement of the subject or both. The X-ray image of the currently healthy subject is as shown in FIG. 3A.

Using a trained identifier 22, the estimation part 12 estimates future lung function of the subject from the lifestyle habit information and the current X-ray image that have been acquired by the acquisition part 11. The output part 13 can output a future X-ray image of the subject as an estimated result. In other words, the estimation part 12 determines an image processing parameter from the lifestyle habit information and the current X-ray image that are used as input to the identifier 22, and the output part 13 uses the determined image processing parameter to alter the X-ray image so that the altered X-ray image may be outputted. The future X-ray image of the subject is as shown in FIG. 3B.

With regards to the estimation, a specific time in the future may be set as deemed appropriate and may be specified via a time parameter that is inputted to the estimation part 12 (the same holds true for Specific Examples 2-4). For example, the acquisition part 11 may acquire the time parameter through an input from a user (e.g., a doctor) who operates the medical diagnosis support system 1.

The X-ray image of FIG. 3B shows features that are characteristic of COPD. That is, in comparison to the X-ray image of FIG. 3A, the lung parts of the image are darker, the lung parts of the image are vertically elongated (as indicated by unshaded arrows), and the heart part of the image is thinner (the width in the horizontal direction is reduced; as indicated by shaded arrows). Thus, the future likelihood of the subject suffering from COPD if the subject was to maintain the current lifestyle habit can thus be conveyed to the subject in a way that is easy to understand. As a result, it becomes possible to encourage the subject to improve the current lifestyle habit, and medical diagnosis support concerning the lung function of the subject is achieved.

### Specific Example 2

In another example, the acquisition part 11 acquires the following information as lifestyle habit information of a currently healthy subject: (a) information on past and present smoking history, and (b) a result of spirometer measurement. A spirogram of the currently healthy subject is as shown by a curve 41 of FIG. 4. From the spirogram, a vital capacity (forced vital capacity; FVC) and a forced expiratory volume in 1 second (FEV1; FEV is an abbreviation for forced expiratory volume) may be derived.

Using a trained identifier 22, the estimation part 12 estimates future lung function of the subject from the lifestyle habit information acquired by the acquisition part 11. The output part 13 can output a future spirogram of the subject as an estimated result. In other words, the estimation part 12 determines an image processing parameter from the lifestyle habit information that is used as input to the identifier 22, and the determined image processing parameter is used by the output part 13 to alter the spirogram so that the altered spirogram may be outputted. A future spirogram of the subject is as shown by a curve 42 of FIG. 4.

Furthermore, the output part 13 may output, as a sample, a spirogram indicating predicted vital capacity that is in accordance with an age, height, and gender of the subject in the future. The spirogram of the predicted vital capacity is as shown by a curve 43 of FIG. 4. In some embodiments, when outputting the curve 43, the age is shifted by the same amount as the time parameter that is inputted to the estimation part 12 while the height and gender of the subject are kept unchanged.

Compared to the curve 41, the curve 42 of FIG. 4 shows features such as reduced inspiratory capacity (the maximum peak shifts downwards as shown by the unshaded arrow) and reduced forced expiratory volume in 1 second (the volume 1 second after maximum volume of air is inhaled shifts upwards as shown by a shaded arrow). Thus, the future likelihood of experiencing reduced lung function if the subject was to maintain the current lifestyle habit can be conveyed to the subject in a way that is easy to understand. As a result, it becomes possible to encourage the subject to improve the current lifestyle habit, and medical diagnosis support concerning the lung function of the subject is achieved.

Also, the curve 42 is seen below curve 43 prior to a forced exhalation. Thus, the future likelihood of experiencing reduced lung function if the subject was to maintain the current lifestyle habit can be conveyed to the subject objectively. As a result, it becomes possible to encourage the subject to improve the current lifestyle habit, and medical diagnosis support concerning the lung function of the subject is achieved.

### Specific Example 3

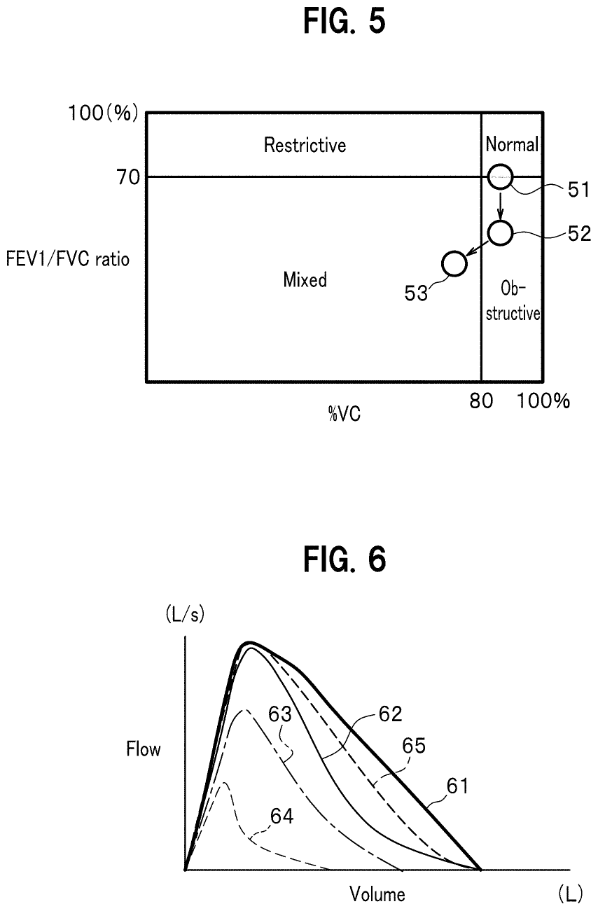

In another example, the acquisition part 11 acquires the following as lifestyle habit information of a currently healthy subject: (a) information on past and present smoking history, and (b) a result of spirometer measurement. The state of lung function of the currently healthy subject is as indicated by a point 51 plotted on the diagnostic reference chart of FIG. 5.

The diagnostic reference chart is a matrix with percentage vital capacity (“% VC”) as the horizontal axis and FEV1/FVC ratio as the vertical axis. The percentage vital capacity (described in percentage [%] in FIG. 5) is a ratio of measured vital capacity (in other words, FVC) to predicted vital capacity. The FEV1/FVC ratio (described in percentage [%] in FIG. 5) is the ratio of FEV1.0 to measured vital capacity.

When the % VC is in a range between 80% and 100% and the FEV1/FVC ratio is in a range between 70% and 100%, lung function is normal (“Normal” of FIG. 5). When the % VC is in a range between 80% and 100% and the FEV1/FVC ratio is equal to or below 70%, lung function indicates an obstructive ventilatory defect (“Obstructive” of FIG. 5). When the % VC is equal to or below 80% and the FEV1/FVC ratio is in a range between 70% and 100%, lung function indicates a restrictive ventilatory defect (“Restrictive” of FIG. 5). When the % VC is equal to or below 80% and the FEV1/FVC ratio is equal to or below 70%, lung function indicates a mixed ventilatory impairment (“Mixed” of FIG. 5) that includes both an obstructive ventilatory defect and a restrictive ventilatory defect.

Using a trained identifier 22, the estimation part 12 estimates future lung function of the subject from the lifestyle habit information acquired by the acquisition part 11. The output part 13 can output a diagnostic reference chart that includes information on a future lung function of the subject as an estimated result. In other words, the estimation part 12 determines an image processing parameter from the lifestyle habit information that is used as input to the identifier 22, and the determined image processing parameter is used by the output part 13 to output a diagnostic reference chart on which a point indicating future lung function is plotted. The future lung function of the subject is as shown by a point 52 in FIG. 5.

According to FIG. 5, the state of lung function is shown to transition from point 51 in the “Normal” region to point 52 in the “Obstructive” region. Thus, the future likelihood of the subject suffering from an obstructive ventilatory defect if the subject was to maintain the current lifestyle habit can be conveyed to the subject in a way that is easy to understand. As a result, it becomes possible to encourage the subject to improve the current lifestyle habit, and medical diagnosis support concerning the lung function of the subject is achieved.

The output part 13 may plot how the state of the lung function of a subject transitions using the diagnostic reference chart of FIG. 5. More specifically, by continuously increasing the time parameter that is used by the estimation part 12 to estimate future lung function, the output part 13 can display a transition in the future state of lung function as a movement of points plotted on the diagnostic reference chart. FIG. 5 shows the plotted points transitioning from point 52 in the “Obstructive” region to point 53 in the “Mixed” region, indicating that the state of lung function transitions from an obstructive ventilatory defect to a mixed ventilatory impairment. The future likelihood of the state of the lung function deteriorating further to a mixed ventilatory impairment if the subject was to maintain the current lifestyle habit can thus be conveyed to the subject in a way that is easy to understand. As a result, it becomes possible to encourage the subject to improve the current lifestyle habit, and medical diagnosis support concerning the lung function of the subject is achieved.

### Specific Example 4

In another example, the acquisition part 11 acquires the following as lifestyle habit information of a currently healthy subject: (a) information on past and present smoking history, and (b) a result of spirometer measurement. A state of lung function of the currently healthy subject is as indicated by a curve 61 (normal) of a flow curve of FIG. 6.

FIG. 6 shows a flow curve showing curves associated with an obstructive ventilatory defect. The x-axis represents the amount of air exhaled or inhaled (synonymous to lung volume) in liters and the y-axis represents the speed (flow) of air in liters per second. An obstructive ventilatory defect is a disorder related to a difficulty in expiration due to obstruction of a peripheral airway. Because flow is reduced due to difficulty in breathing out, the curve is characterized by a concave upward pattern during a fall in the flow. In the case of a mild impairment (curve 62), although there is no drop in volume, the curve is characterized by a concave upward pattern during a fall in the flow. In the case of a moderate impairment (curve 63), the curve is characterized by a drop in volume and a concave upward pattern during a fall in the flow. In the case of a severe impairment (curve 64), the curve shows a significant drop in both volume and flow, which is a characteristic often observed in individuals with severe COPD.

Using a trained identifier 22, the estimation part 12 estimates future lung function of the subject from the lifestyle habit information acquired by the acquisition part 11. The output part 13 can output a future flow curve of the subject as an estimated result. In other words, the estimation part 12 determines an image processing parameter from the lifestyle habit information that is used as input to the identifier 22, and the determined image processing parameter is used by the output part 13 to output a flow curve indicating the future lung function. The flow curve of the future lung function of the subject is as shown by a curve 65 in FIG. 6.

According to FIG. 6, the flow curve of the subject moves away from “normal” toward “mild impairment”. The curve 65 shows some concave upward pattern during a fall in the flow. Thus, the future likelihood of the subject suffering from an obstructive ventilatory defect if the subject was to maintain the current lifestyle habit can be conveyed to the subject in a way that is easy to understand. As a result, it becomes possible to encourage the subject to improve the current lifestyle habit, and medical diagnosis support concerning the lung function of the subject is achieved.

### Hardware Configuration

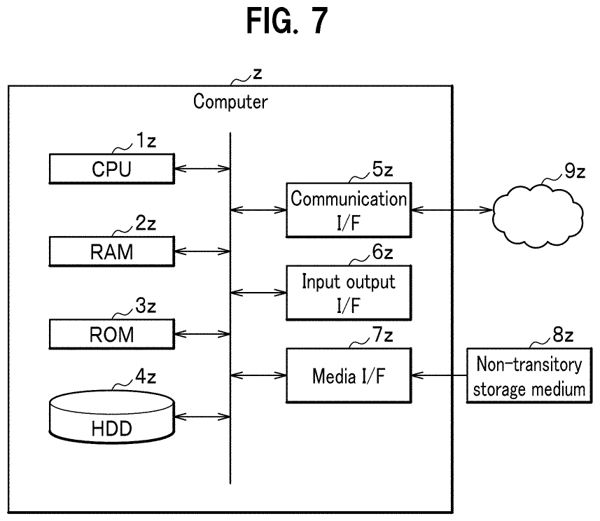

The abovementioned medical diagnosis support system 1 may, for example, be achieved using a computer z with a hardware configuration of FIG. 7. The computer z includes a CPU 1z (a central processing unit 1z), RAM 2z (random-access memory 2z), ROM 3z (read-only memory 3z), HDD 4z (a hard disk drive 4z), a communication interface 5z (a communication I//F 5z), an input output interface 6z (an input output I/F 6z) and a media interface 7z (a media I/F 7z).

The CPU 1z operates based on a program that is stored in the ROM 3z or HDD 4z, and controls individual components (including the acquisition part 11, estimation part 12, output part 13, and control part 14) of the medical diagnosis support system 1. The ROM 3z stores therein a boot program that is executed by the CPU 1z when the computer z is turned on. The ROM 3z also stores therein a program that is dependent on a hardware of the computer z.

The HDD 4z stores therein a program that is executed by the CPU 1z and data and the like that are used by the program. The communication interface 5z is configured to receive data from another device via a communication network 9z, send the received data to the CPU 1z, and send data that is generated by the CPU 1z to another device via the communication network 9z.

The CPU 1z controls, via the input output interface 6z, an output device such as a display, a printer, or the like. The CPU 1z controls, via the input output interface 6z, an input device such as a keyboard, a mouse, or the like. The CPU 1z may acquire data from an input device via the input output interface 6z. The CPU 1z may output generated data to an output device via the input output interface 6z.

The media interface 7z is configured to read out data or a program stored in a non-transitory storage medium 8z and provide that data or program to the CPU 1z via the RAM 2z. The CPU 1z executes the program by loading the program from the non-transitory storage medium 8z onto the RAM 2z via the media interface 7z. A non-transitory storage medium 8z may for example be an optical recording medium such as a DVD (a digital versatile disc) or a PD (a phase-change rewritable disk), a magneto-optical recording medium such as an MO (a magneto-optical disk), a tape medium, a magnetic recording medium, or a semiconductor memory.

For example, in the case where the computer z functions as the medical diagnosis support system 1, the CPU 1z of the computer z executes a program that has been loaded onto the RAM 2z to realize functions of the individual components of the medical diagnosis support system 1. When the program is executed, data and the like that are stored in the HDD 4z are used. In some embodiments, the CPU 1z of the computer z reads out the program to be executed from the non-transitory storage medium 8z. In some embodiments, the program to be executed may be acquired from another device via a communication network 9z.

### Aspects of the Disclosure

According to a second aspect of the disclosure, the medical diagnosis support system is in accordance with the first aspect, the acquisition part is further configured to acquire X-ray image information of the subject, and the information on the subject from which the future lung function is estimated further includes the acquired X-ray image information.

According to a third aspect of the disclosure, a medical diagnosis support system is in accordance with the first or second aspect, and the lifestyle habit information includes at least one of information on a smoking habit of the subject or a result of spirometer measurement.

According to a fourth aspect of the disclosure, a medical diagnosis support system is in accordance with the first, second, or third aspect, and the output part is configured to output at least one of a spirogram, a diagnostic reference chart, a flow curve, or an image.

According to a sixth aspect of the disclosure, the non-transitory computer readable storage medium is in accordance with the fifth aspect, the operations further include acquiring X-ray image information of the subject, and the information on the subject from which the future lung function is estimated further includes the X-ray image information that is acquired.

According to a seventh aspect of the disclosure, a non-transitory computer readable storage medium is in accordance with the fifth or sixth aspect, and the lifestyle habit information includes at least one of information on a smoking habit of the subject or a result of spirometer measurement.

According to an eight aspect of the disclosure, a non-transitory computer readable storage medium is in accordance with the fifth, sixth, or seventh aspect, and output of the output part includes at least one of a spirogram, a diagnostic reference chart, a flow curve, or an image.

According to a tenth aspect of the disclosure, the medical diagnosis support method is in accordance with the ninth aspect, the method further includes acquiring X-ray image information of the subject, and the information on the subject from which the future lung function is estimated further includes the X-ray image information that is acquired.

According to an eleventh aspect of the disclosure, a medical diagnosis support method is in accordance with the ninth or tenth aspect, and the lifestyle habit information includes at least one of information on a smoking habit of the subject or a result of spirometer measurement.

According to a twelfth aspect of the disclosure, a medical diagnosis support method is in accordance with the ninth, tenth, or eleventh aspect, and output of the output part includes at least one of a spirogram, a diagnostic reference chart, a flow curve, or an image.

### Advantageous Effects of Embodiments

According to the disclosure, support for medical diagnosis concerning the lung function of a subject is achieved.

According to an embodiment of the disclosure, an estimated result of future lung function of a subject in a case where the subject was to maintain the current lifestyle habit can be conveyed to the subject in a way that is easy to understand (such as visually) to encourage the subject to review the current lifestyle habit. Support for medical diagnosis concerning the lung function of the subject is therefore achieved.

In some embodiments, X-ray image information of a subject is inputted and a future X-ray image of the subject is outputted as an estimated result. This makes it possible for an estimated result of the future lung function to be conveyed to the subject in a way that is easy to understand.

In some embodiments, at least one of (a) information on a smoking habit of a subject or (b) a result of spirometer measurement of a subject is acquired as lifestyle habit information. This makes it possible for an estimated result of lung function caused by smoking to be conveyed to the subject in a way that is easy to understand. Encouraging the subject to review the smoking habit becomes possible as a result.

In some embodiments, by outputting at least one of a spirograph, a diagnostic reference chart, a flow curve, or an image, an estimated result of the lung function of the subject can be conveyed in a way that is easy to understand.

### First Modification to the Embodiment

In the embodiments described above, future lung function of a subject is estimated when information on a current lifestyle habit of the subject is inputted. In some embodiments, the estimation part 12 may be used to estimate future lung function that is derived by inputting fictional lifestyle habit information that is different to the current lifestyle habit information of a subject. By trying out fictional lifestyle habit information as input, it becomes possible to search for lifestyle habit information that will achieve future lung function with a mitigated or no disorder. It thus becomes possible to advise the subject to review a current lifestyle habit and to switch to a lifestyle habit that is in accordance with the lifestyle habit information obtained through searching.

### Second Modification to the Embodiment

In the embodiments described above, lung function is estimated using an identifier 22 that has been trained using training data that includes (a) lifestyle habit information of an individual prior to becoming affected by a disorder (or lifestyle habit information of a healthy individual) as input data, and (b) a test result (a result of spirometer measurement, X-ray image information, an image processing parameter) of an individual with a lung function disorder as output data. In some embodiments, a different identifier 22 may be used that has been trained using training data that includes (a) a test result of an individual with a lung function disorder as input data, and (b) lifestyle habit information of an individual who has recovered from a lung function disorder (or an individual whose lung function disorder can be mitigated) as output data. By using the different identifier 22, lifestyle habit information that enables a recovery from (or mitigation of) a lung function disorder can be estimated.

### Third Modification to the Embodiment

In some embodiments, the estimation part 12 may output a predetermined type of image indicating future lung function of a subject from lifestyle habit information of a subject that is not image information (the lifestyle habit information may for example be various types of test data sets in the form of numerical values) through the use of a similar image of the predetermined type. For example, the predetermined type of a similar image may be but is not limited to image information of any individual whose gender, age bracket, and height are the same as those of the subject.

### Fourth Modification to the Embodiment

Specific Example 4 has been described in relation to an obstructive ventilatory defect. However, the aspect of the Specific Example 4 is not limited to the obstructive ventilatory defect and may for example be applied to a restrictive ventilatory defect or an upper respiratory tract disorder.

### Fifth Modification to the Embodiment

Various aspects of the disclosure that have been described may be combined as deemed appropriate.

Although embodiments of the present invention have been described and illustrated in detail, the disclosed embodiments are made for purposes of illustration and example only and not limitation. Modifications and variations of the embodiments described above will occur to those skilled in the art in light of the above teachings. The scope of the present invention should be interpreted by terms of the appended claims.

What is claimed is:

1. A medical diagnosis support system comprising
a hardware processor configured with a program to perform operations comprising:
operation as an acquisition part configured to acquire lifestyle habit information of a subject;
operation as an estimation part configured to estimate, using a trained identifier, future lung function of the subject from information on the subject, the information on the subject including the lifestyle habit information acquired from the acquisition part; and
operation as a control part configured to control an output part to output a result that has been estimated by the estimation part.

2. The medical diagnosis support system according to claim 1, wherein
the acquisition part is further configured to acquire X-ray image information of the subject, and
the information on the subject from which the future lung function is estimated further includes the acquired X-ray image information.

3. The medical diagnosis support system according to claim 1, wherein
the lifestyle habit information includes at least one of information on a smoking habit of the subject or a result of spirometer measurement.

4. The medical diagnosis support system according to claim 1, wherein
the output part is configured to output at least one of a spirogram, a diagnostic reference chart, a flow curve, or an image.

5. A non-transitory computer readable storage medium storing a medical diagnosis support program executable by a computer, the medical diagnosis support program causing the computer to perform operations of a medical diagnosis support system, the operations comprising:
acquiring lifestyle habit information of a subject;
estimating, using a trained identifier, future lung function of the subject from information on the subject, the information on the subject including the lifestyle habit information that has been acquired; and
controlling an output part to output a result that has been estimated.

6. The non-transitory computer readable storage medium of claim 5, wherein
the operations further comprise acquiring X-ray image information of the subject, and
the information on the subject from which the future lung function is estimated further includes the X-ray image information that is acquired.

7. The non-transitory computer readable storage medium according to claim 5, wherein
the lifestyle habit information includes at least one of information on a smoking habit of the subject or a result of spirometer measurement.

8. The non-transitory computer readable storage medium according to claim 5, wherein
output of the output part includes at least one of a spirogram, a diagnostic reference chart, a flow curve, or an image.

9. A medical diagnosis support method used in a medical diagnosis support system, the medical diagnosis support method comprising:
acquiring lifestyle habit information of a subject;
estimating, using a trained identifier, future lung function of the subject from information on the subject, the information on the subject including the lifestyle habit information that has been acquired; and
controlling an output part to output a result that has been estimated.

10. The medical diagnosis support method of claim 9, wherein
the method further comprises acquiring X-ray image information of the subject, and
the information on the subject from which the future lung function is estimated further includes the X-ray image information that is acquired.

11. The medical diagnosis support method according to claim 9, wherein
the lifestyle habit information includes at least one of information on a smoking habit of the subject or a result of spirometer measurement.

12. The medical diagnosis support method according to claim 9, wherein
output of the output part includes at least one of a spirogram, a diagnostic reference chart, a flow curve, or an image.
